(19) United States
(12) Patent Application Publication
(10) Pub. No.: US 2026/0207659 A1
(43) Pub. Date: Jul. 23, 2026

(54) COMPOSITIONS FOR USE IN THE PROPHYLACTIC TREATMENT OF HYPOCALCEMIA IN A RUMINANT ANIMAL

(71) Applicant: VILOFOSS A/S, Fredericia (DK)

(72) Inventor: Per THEILGAARD, Grasten (DK)

(21) Appl. No.: 19/137,702

(22) PCT Filed: Dec. 12, 2022
(86) PCT No.: PCT/EP2022/085402

§ 371 Date: Jun. 11, 2025

(51) Int. Cl. A61K 33/244; A61K 31/14; A61K 31/785; A61K 33/00; A61K 33/04; A61K 33/06; A61K 33/08; A61K 33/14; A61K 33/18; A61K 33/24; A61K 33/26; A61K 33/30; A61K 33/32; A61K 33/34; A61P 3/14

(52) U.S. Cl. CPC A61K 33/244; A61K 31/14; A61K 31/785; A61K 33/00; A61K 33/04; A61K 33/06; A61K 33/08; A61K 33/14; A61K 33/18; A61K 33/24; A61K 33/26; A61K 33/30; A61K 33/32; A61K 33/34; A61P 3/14

(57) ABSTRACT

Compositions and principles for use before parturition in the prophylactic treatment of hypocalcemia in ruminant animals include compositions having phosphate binders for the prophylactic treatment of parturient hypocalcemia in ruminant animals. These compositions are presented in unit dosage forms, with one or more doses administered daily to the gastrointestinal tract of the ruminant animal, or in wet form. The composition contains one or more phosphate binders and one or more nutrients selected from minerals, vitamins, and amino acids, as well as a composition that includes an inhibitor of the small intestinal Na-phosphate transporter for the prophylactic treatment of parturient hypocalcemia in ruminant animals. Phosphate binders are selected from aluminum, iron, sevelamer, and lanthanum salts, with either inorganic or organic acids, hydrates thereof, or any mixture thereof.

### FIELD OF THE INVENTION

The present invention relates to compositions and principles for use before parturition in the prophylactic treatment of hypocalcemia in ruminant animals.

### BACKGROUND OF THE INVENTION

Parturient hypocalcemia is a common disease in dairy cows. Incidents of clinical hypocalcemia (milk fever) amount to around 4% worldwide with big between farm variations. Subclinical clinical hypocalcemia usually occurs on 25% on first calves and 50% for multiparous cows.

Parturient hypocalcemia occurs when the cow is not able to sustain the plasma calcium level due to demands for calcium. The calcium level in plasma is usually strictly homeostatically regulated and is usually very constant across different metabolic stages of the reproductive cycle. Parturient hypocalcemia usually occurs in the transition period from dry to lactating cows. During the dry period the demand for calcium is very low and the absorption mechanism of calcium is not activated as the demand of calcium can be undertaken from the passive absorption between epithelia cells. Close to calving, the animals start to produce colostrum and later milk initiating a sudden increased demand of calcium. The plasma calcium level decreases initiating the cow's own defence mechanism in order to counteract the plasma calcium drop. The time lag for starting the active absorption mechanism is though several days impairing the production capacity of the animal. The production response of parturient hypocalcemia is well described and includes increased frequency of dystocia, retained placenta, metritis, ketosis, lower reproduction and increased culling rate.

The existing methods for preventing parturient hypocalcemia include:
Dietary cation anion balance (DCAB) in the pre-calving diet inducing a controlled metabolic subclinical acidosis; the cow counteracts this by releasing more calcium to the plasma and thereby starting the cow's own absorption mechanism of calcium;
High level of zeolite in the feed;
Low calcium feeding in the pre-calving diet activates the cow's absorption mechanism; and
High calcium feeding in the periparturient diet—increases the passive absorption of calcium sustaining the plasma calcium level.

EP 1 162 890 B1 discloses the use of at least one compound which reduces absorption of calcium from the drinking water and/or for the ration of a cow for preventing parturient hypocalcemia in a cow. Various compounds for this use are suggested, e.g. oxalic acid, sodium oxalate, phytic acid, a phytate, a clay mineral including zeolite, ethylenediaminetetraacetic acid (EDTA) and its sodium salts Na₂EDTA and Na₄EDTA, trisodium nitrilotriacetate monohydrate, trisodium nitriloacetate, pentasodium diethylenetriaminepentaacetate, trisodium N-hydroxyethyl-ethylenediaminetriacetate, citric acid, a citrate, a polyphosphate, a tripolyphosphate, an orthophosphate and a cellulose phosphate and a calcium-free derivative of any such compounds, as well as a zinc compound selected from zinc oxide, zinc chloride and zinc sulphate.

WO 2008/005217 A2 discloses pharmaceutical compositions comprising a pharmaceutically acceptable ferrous iron compound for use in a method of treating a subject with hyperphosphatemia.

U.S. Pat. No. 8,236,358 B1 discloses compositions and methods suitable for the treatment of hyperphosphatemia based on phosphate-binding magnesium salts.

U.S. Pat. No. 4,931,290 discloses a method for reducing the propensity of a dairy cow to develop severe milk fever upon calving comprising administering to the cow after calving a composition comprising a water-soluble calcium compound and a complexing agent for serum phosphorus.

Pallesen et al.: “Effect of pre-calving zeolite, magnesium and phosphorus supplementation on periparturient serum mineral concentrations”, VETERINARY JOURNAL, BAILLIERE TINDALL, LONDON, GB, vol. 175, no. 2, 1 Feb. 2008, p. 234-239, XP022491628, ISSN: 1090-0233, DOI: 10.1016/J.TVJL.2007.01.007, discloses a study to test whether supplementing dry cow rations with phosphorus and magnesium would interfere with the beneficial effect of zeolite supplementation on the periparturient blood calcium concentration in dairy cattle.

There is still a need for a composition for use before parturition in the prophylactic treatment of parturient hypocalcemia in a ruminant animal, e.g. a cow.

### SUMMARY OF THE INVENTION

In a first aspect, the present invention provides a composition comprising one or more phosphate-binders for use before parturition in the prophylactic treatment of parturient hypocalcemia in a ruminant animal, cf. claim 1.

In a second aspect, the present invention provides a composition comprising one or more phosphate-binders for use before parturition in the prophylactic treatment of parturient hypocalcemia in a ruminant animal, wherein the composition is in unit dosage form(s), and wherein one or more dosages per day of the unit dosage form(s) of the composition are manually administered to the gastrointestinal tract of the ruminant animal, cf. claim 12.

In a third aspect, the present invention provides a composition comprising one or more phosphate binders for use before parturition in the prophylactic treatment of parturient hypocalcemia in a ruminant animal, wherein the composition is presented in wet form, cf. claim 15.

### LEGENDS TO THE FIGURE

FIG. 1 shows the effect of the phosphate binder aluminium sulphate on the serum inorganic phosphate level in the periparturient period for a control and treatment group, respectively;

FIG. 2 shows the effect of the phosphate binder aluminium sulphate on the serum total calcium level in the periparturient period for a control and treatment group, respectively; and

FIG. 3 shows the effect of the phosphate binder aluminium sulphate on the milk yield in the first 5 weeks after parturition for a control and treatment group, respectively; for the experiment described in more detail in EXAMPLE 2 below.

### DETAILED DESCRIPTION OF THE INVENTION

A number of improvements and alternatives to the prophylactic treatment of parturient hypocalcemia are described in the following. The improvements and alternatives primarily take advantage of the use of phosphate-binders.

In the present context, the term “phosphate-binder” is to be understood as referring to a compound or material capable of binding phosphate, e.g. typically in an amount of at least 50 mg P/g, in particular at least 100 mg P/g, in particular at least 150 mg P/g.

Examples of phosphate-binders are aluminium citrate, aluminium chloride, aluminium carbonate, aluminium sulphate, aluminium oxide, aluminium ammonium sulphate, aluminium sodium sulphate, aluminium potassium sulphate, aluminium calcium silicate, aluminium silicate, aluminium hydroxide, aluminium bromide, aluminium iodide, aluminium nitrate, sodium aluminium phosphate, lanthanum carbonate, lanthanum carbonate hydroxide, lanthanum oxycarbonate, lanthanum chloride, ferrous citrate, ferrous sulphate, ferrous citrate, ferrous chloride, ferrous carbonate, ferrous bicarbonate, ferric citrate, ferric sulphate, ferric chloride, ferric ammonium citrate, sucroferric oxyhydroxide, ferric trimaltol, ferric bicarbonate, ferric carbonate, sevelamer hydrochloride, sevelamer carbonate, a hydrate thereof or any mixture thereof. Preferably, the applicable phosphate-binder are non-toxic at the therapeutically effective amounts.

In the present context, the term “ruminant animals” typically refers to cows, sheep and goats, in particular cows.

### Phosphate-Binders in the Prophylactic Treatment of Parturient Hypocalcemia

Several works have investigated the relationship between the inorganic phosphor level in the pre-calving diet and the parturient hypocalcemia. Phosphate may also play an important role in the pathogenesis of milk fever, with increasing phosphate concentrations increasing milk fever risk. In cattle, there is evidence that a pre-calving diet high in phosphate can have a negative impact on calcium homeostasis at calving (Kichura, et al., 1982, Livestock Prod. Sci., 31:271-286; Barton, et al., 1987, J. Dairy Sci. 70:1186-1191). It is therefore recommended that the pre-calving diet includes a maximum of 22 g phosphorous per animal per day (National Research Council. 2001. Nutrient Requirements of Dairy Cattle. 7^{th} revised edition. National Research Council. National Academic Press, Washington, DC.; Weiss, Real World Recommendations for Minerals and Vitamins. Penn State Dairy Cattle Workshop November 12-14 Nov. 2012. 107-112). Due to the natural occurrence of phosphorous in commercial feed ingredients and home grown feed the phosphorous level usually is around 35 to 50 gram per day in the diet. It can therefore not be used under practical farm conditions.

The present inventors have found that reducing phosphorus is an efficient method for preventing parturient hypocalcemia. Under normal practice it is though impossible/difficult to reach a low amount of phosphorus in the diet due to the phosphor level in the normal feed ingredients used.

EP 1162890 B1 discloses the use of a zeolite for preventing parturient hypocalcemia. The reference exclusively deals with the calcium-binding effect of the zeolite.

However, the present inventors have found that phosphate-binding has a more significant effect than calcium-binding in the prophylactic treatment of parturient hypocalcemia.

Also, the current use of synthetic sodium aluminosilicate zeolite type A (a calcium-binder as well as a phosphate-binder) for the prophylactic treatment of parturient hypocalcemia requires significant amounts of the material (about 400 g/cow/day) to be administered, and because it has been found that phosphate-binding has a more significant effect than calcium-binding, it is believed to be advantageous to use a designated phosphate-binder alone, or alternatively in combination with e.g. a zeolite, in the prophylactic treatment of parturient hypocalcemia.

Therefore, in a first aspect, the present invention provides a composition comprising one or more phosphate-binders selected from aluminium, iron, sevelamer, and lanthanum salts with inorganic or organic acids, a hydrate thereof or any mixture thereof for use before parturition in the prophylactic treatment of parturient hypocalcemia in a ruminant animal. Thereby subclinical or clinical hypocalcemia in the peri-parturient cow can be reduced or avoided.

In one variant hereof, the composition further comprises a zeolite, e.g. a synthetic sodium aluminosilicate zeolite A type. In such variants, the weight ratio of the one or more phosphate-binders to the zeolite is in the range of 99:1 to 10:90. By incorporating also a zeolite in the composition according to the invention both a phosphate-binding and a calcium-binding effect in the prophylactic treatment of parturient hypocalcemia is obtained.

### Phosphate-Binder and Nutrients

Due to an imbalance in the physiological system shortly before and shortly after the time of calving, cows may suffer from deficiencies with respect to certain minerals, vitamins, amino acids and related substances. Although addition of certain supplements to the regular animal feed is a possibility, it will require the formulation of a particular quality of feed to be administered to the cows around the time of calving, in particular shortly before calving. Because it may be desirable to administer a composition comprising a phosphate-binder to the cows the last four weeks before calving, such as the last two-three weeks before calving, such as the last 1-2 weeks before calving, such as a least in the period 1-10 days before parturition for the purpose of the prophylactic treatment of hypocalcemia, it is for practical reasons and for the reasons of accurate administration believed to be advantageous to co-administer to cows a phosphate-binder in combination with certain nutrients, like minerals, vitamins and amino acids.

Therefore, the present invention also provides a composition for use before parturition in the prophylactic treatment of parturient hypocalcemia in a ruminant animal further comprising one or more nutrients selected from minerals, vitamins and amino acids.

In an embodiment the minerals, vitamins and amino acids are selected from choline, magnesium oxide, magnesium chloride, magnesium sulphate, sodium chloride, sodium bicarbonate, sodium sulphate, selenium chloride, sodium selenite, cobalt chloride, cobalt sulphate, iodine chloride, copper chloride, copper sulphate, manganese chloride, manganese sulphate, manganese oxide, zinc chloride, zinc sulphate, zinc oxide, vitamin A, vitamin D, vitamin E, vitamin B12, pantothenic acid, folic acid, thiamine, biotin, niacin, and lysine, isoleucine, threonine, lysine and rumen protected methionine, respectively, such as, niacin and rumen protected methionine, respectively.

Such minerals, vitamins and amino acids are typically included in a total amount of 10 to 500 gram per daily dose of the composition.

Typically, the one or more phosphate-binders are selected from aluminium citrate, aluminium chloride, aluminium carbonate, aluminium sulphate, aluminium oxide, aluminium ammonium sulphate, aluminium sodium sulphate, aluminium potassium sulphate, aluminium calcium silicate, aluminium silicate, aluminium hydroxide, aluminium bromide, aluminium iodide, aluminium nitrate, sodium aluminium phosphate, lanthanum carbonate, lanthanum chloride, iron citrate, iron sulphate, sevelamer hydrochloride, sevelamer carbonate, a hydrate thereof or any mixture thereof, preferably selected from the group consisting of lanthanum chloride, aluminium chloride, aluminium sulphate, iron (II) sulphate, sevelamer hydrochloride, lanthanum carbonate, a hydrate thereof or a mixture thereof.

In one embodiment, the composition furthermore comprises a zeolite.

The composition may be useful before parturition in the prophylactic treatment of parturient hypocalcemia in a ruminant animal, e.g. a cow.

### Large Unit Dosage Forms of Phosphate-Binders

By the administration of a phosphate-binder in granulate form to a ruminant animal, such as a cow, the desired amount of a granulate comprising the phosphate-binder may be top-dressed onto the regular animal feed. However, the ingested amount of the phosphate-binder granulate by the cow will depend on whether the granulate stays homogeneously in admixture with the feed, or whether a portion of the granulate sinks through the feed and is left behind after the cow has ingested the feed.

The present inventors have found that the manual administration of “a bolus” (possibly presented as a number of unit dosage forms) to the cow will ensure accurate administration of the phosphate-binder to the cow.

Therefore, in a second aspect, the present invention provides a composition comprising one or more phosphate-binders for use in the prophylactic treatment of parturient hypocalcemia in a ruminant animal, e.g. a cow, wherein the composition is in unit dosage form(s) each comprising 100-800 g of the phosphate-binder, such as 150-700, such as 200-600, such as 250-500, such as 300-400 g of the phosphate binder, and wherein one or more dosages per day of the unit dosage form(s) of the composition are manually administered to the gastrointestinal tract of the ruminant animal, e.g. the cow.

Typically, the one or more phosphate-binders are selected from aluminium citrate, aluminium chloride, aluminium carbonate, aluminium sulphate, aluminium oxide, aluminium ammonium sulphate, aluminium sodium sulphate, aluminium potassium sulphate, aluminium calcium silicate, aluminium silicate, aluminium hydroxide, aluminium bromide, aluminium iodide, aluminium nitrate, sodium aluminium phosphate, lanthanum carbonate, lanthanum chloride, iron citrate, iron sulphate, sevelamer hydrochloride, sevelamer carbonate, a hydrate thereof or any mixture thereof, preferably selected from the group consisting of lanthanum chloride, aluminium chloride, aluminium sulphate, iron (II) sulphate, sevelamer hydrochloride, lanthanum carbonate, a hydrate thereof or a mixture thereof.

In one embodiment, the composition furthermore comprises a zeolite.

In some embodiments, the composition further comprises one or more nutrients as described under the heading “Phosphate-binder and nutrients” above.

### Composition of Phosphate-Binder in Wet Form

With respect to the challenges described under the heading “Large unit dosage forms of phosphate-binders”, it is believed that the formulation of the phosphate-binder in moistened or water-suspended form will make the composition of the phosphate-binder capable of efficiently adhering to the regular animal feed whereby only an insignificant portion of the phosphate-binder will run through the feed and be left behind after the cow has ingested the feed.

Therefore, in a third aspect, the present invention provides a composition comprising one or more phosphate binders for use before parturition in the prophylactic treatment of parturient hypocalcemia in a ruminant animal, e.g. a cow, wherein the composition is presented to the animal in wet form comprising 15-80%, e.g. 20-50%, by weight of water.

Typically, the one or more phosphate-binders are selected from aluminium citrate, aluminium chloride, aluminium carbonate, aluminium sulphate, aluminium oxide, aluminium ammonium sulphate, aluminium sodium sulphate, aluminium potassium sulphate, aluminium calcium silicate, aluminium silicate, aluminium hydroxide, aluminium bromide, aluminium iodide, aluminium nitrate, sodium aluminium phosphate, lanthanum carbonate, lanthanum carbonate hydroxide, lanthanum oxycarbonate, lanthanum chloride, ferrous citrate, ferrous sulphate, ferrous citrate, ferrous chloride, ferrous carbonate, ferrous bicarbonate, ferric citrate, ferric sulphate, ferric chloride, ferric ammonium citrate, sucroferric oxyhydroxide, ferric trimaltol, ferric bicarbonate, ferric carbonate, sevelamer hydrochloride, sevelamer carbonate, a hydrate thereof or any mixture thereof, such as selected from the group consisting of aluminium citrate, aluminium chloride, aluminium sulphate, lanthanum chloride, lanthanum carbonate hydroxide, lanthanum oxycarbonate, ferrous citrate, ferrous chloride, ferrous carbonate, ferrous bicarbonate, ferrous sulphate, ferric citrate, ferric sulphate, ferric chloride, ferric ammonium citrate, sevelamer hydrochloride, sevelamer carbonate, preferably selected from the group consisting of lanthanum chloride, aluminium chloride, aluminium sulphate, ferrous sulphate, sevelamer hydrochloride, lanthanum carbonate, a hydrate thereof or a mixture thereof.

In one embodiment, the composition furthermore comprises a zeolite.

In some embodiments, the composition further comprises one or more nutrients as described under the heading “Phosphate-binder and nutrients” above.

### EXAMPLES

### Example 1: Phosphorus Binding Efficacy

Experiments were conducted to assess the phosphorus binding efficacy of aluminium, iron, sevelamer, and lanthanum compounds in comparison to other compounds. The in vitro method used is described by Thilsing et al. (Thilsing T, et al. Journal of Veterinary Medicine Series A. 53(2), 57-64 (2006), the entire contents of which is herein incorporated by reference), which method mimics the digestive tract of a ruminant animal.

Briefly, the experiment was conducted in three consecutive phases. In phase 1, rumen fluid with or without one of the test compounds was incubated at pH typical in the rumen (pH about 7-8). In phase 2, HCl was added to mimic the abomasal conditions (pH about 1.5-3.5) and incubation was resumed. In phase 3, the pH was increased with HCO₃⁻ to mimic conditions in the small intestine (pH about 6.8-7.2) and incubation was continued. The amount of each compound added to the rumen fluid (0.06 g/8 ml of rumen fluid) corresponds to the amounts which would be used in practice, e.g., 600 g of compounds in approximately 100 kg of ingesta). The amount of compound was not adjusted for dry matter or other.

The free phosphorus (P) across the three phases for each compound tested is shown in Table 1. Thus, the lower the concentration of P, the more phosphorus has been absorbed by the compound in question. Table 1 also compares the amount of free P left by each compound to the amount of free P in the Control samples where no test compound was used and left for Zeolite 4A.

When converted to binding capacity in milligram P per gram of tested compound, the results are tabulated in Table 2. These values are all from phase 3, representing the small intestine the ruminant, which is main site for phosphorus absorption.

As seen in Table 1 and Table 2, simple lanthanum, aluminium, iron and sevelamer compounds such as lanthanum chloride, lanthanum carbonate, aluminium chloride, aluminium sulphate, iron (II) sulphate, and sevelamer hydrochloride are superior phosphorus binders to many other compounds, including being superior to the use of Zeolite 4A alone. Because the compounds are generally well tolerated physiologically, they are excellent for use in feed compositions for lactating animals.

Table 1. Phosphate concentration of the supernatant in the Ca- and/or P-enriched rumen fluid in samples without (Control) and with the addition of various compounds after addition of HCl and HCO3⁻. Phosphate content is compared to synthetic zeolite 4A and control.

| | | Phosphate | Phosphate |
|---|---|---|---|
| | Free | binding in | binding in |
| | Phosphate | comparison to | comparison to |
| Compound | (mmol) | 4A, % | control, % |
| Lanthanum chloride | 6.3 | 51.1 | 67.9 |
| Aluminium chloride | 7.0 | 45.6 | 64.3 |
| Iron (II) sulphate | 8.2 | 36.5 | 58.3 |
| Fe₂SO₄ − 6-7H₂0 | | | |
| Sevelamer hydrochloride | 10.0 | 22.8 | 49.3 |
| Lanthanum carbonate, hydrated | 10.0 | 22.6 | 49.2 |
| Aluminium sulphate | 10.3 | 20.1 | 47.5 |
| Zeolite 4A (Zeocros ™ CA 150) | 12.9 | 0.0 | 34.3 |
| Untreated control | 19.7 | −52.3 | 0.0 |

Table 2. Binding capacity of inorganic phosphate at phase 3 (after addition of HCl and HCO⁻—mimicking small intestine) for various compounds. Binding capacity of the compound in question is compared to binding capacity of synthetic 4A and control.

| | Phosphate Binding | Phosphate | |
|---|---|---|---|
| | Capacity(mg | binding in | P binding in |
| | Phosphate/gram of | comparison to | comparison to |
| Compound | compound) | 4A, % | control, % |
| Aluminium chloride | 88.9 | 94 | 96 |
| Lanthanum carbonate, | 77.7 | 72 | 84 |
| hydrated | | | |
| Lanthanum chloride | 72.8 | 62 | 79 |
| Iron (II) sulphate | 69.3 | 56 | 75 |
| Aluminium sulphate | 68.3 | 54 | 74 |
| Sevelamer hydrochloride | 46.5 | 11 | 50 |
| Zeolite 4A (Zeocros ™ CA | 40.8 | 0 | 44 |
| 150) | | | |
| Untreated control | 0.0 | −79 | 0 |

### Example 2: Effect of Compounds on Reducing Hypocalcaemia in Peripartum Dairy Cows

34 multiparous cows were enrolled 21 d prior to calving and randomly assigned to 1 of 2 dietary treatments. Control treatment (n=17); corn silage based diet typical in the industry. Experimental cows (n=17) same ration+400 gram aluminium sulphate pr day.

Blood samples were taken from the cows according to the following schedule:
at the day of grouping into the close-up group—baseline values;
10, 7, 5, 3 and 1 days before the expected calving;
at the day of calving (within 12 hours);
1, 2, 3, 5, 7, and 14 days after calving.

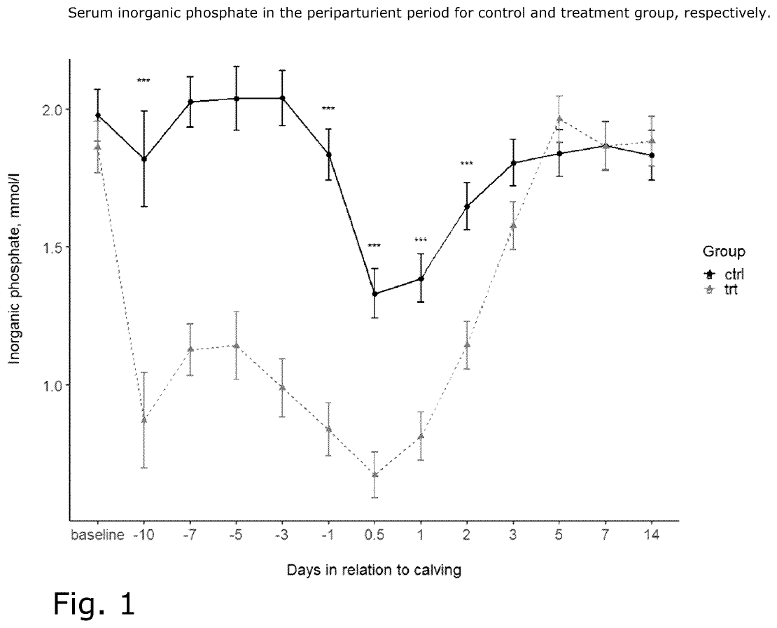

Supplementation of aluminium sulphate significantly (P<0.001) decreased the concentration of inorganic phosphate from the pre-start value of around 2 mmol/L to a level around 0.84 mmol/L at parturition, whereas the control values were at 1.84 mmol/L. This demonstrates a high efficiency reducing the inorganic phosphate (Table 3a & 3b and FIG. 1). After parturition, where the aluminium sulphate no longer was supplemented, the plasma concentration increased to the same level of plasma inorganic phosphate as control after 3-5 days, showing that the decrease is only transient.

Table 3a & 3b. Concentration of plasma inorganic phosphate in the periparturient. The standard error of the mean (SEM) for the entire control group was 0.1 and for the entire treatment group was 0.1. SEM difference between the entire control group and the entire treatment group was 0.14. P-value shown for contrast test between control and treatment.

TABLE 3a

| | Day from calving | | | | | |
|---|---|---|---|---|---|---|
| | baseline | −10 | −7 | −5 | −3 | −1 |
| Control | 1.98 | 1.82 | 2.03 | 2.04 | 2.04 | 1.84 |
| (mmol/L) | | | | | | |
| Treatment | 1.86 | 0.87 | 1.13 | 1.14 | 0.99 | 0.84 |
| (mmol/L) | | | | | | |
| Difference | 0.12 | 0.95 | 0.90 | 0.90 | 1.05 | 1.00 |
| (mmol/L) | | | | | | |
| P-value | 0.378 | <0.001 | <0.001 | <0.001 | <0.001 | <0.001 |
| (pairwise) | | | | | | |

TABLE 3b
(continued from table 3a).

| | Day from calving | | | | | | | |
|---|---|---|---|---|---|---|---|---|
| | baseline | 0.5 | 1 | 2 | 3 | 5 | 7 | 14 |
| Control | 1.98 | 1.33 | 1.39 | 1.65 | 1.81 | 1.84 | 1.87 | 1.83 |
| (mmol/L) | | | | | | | | |
| Treatment | 1.86 | 0.67 | 0.81 | 1.15 | 1.58 | 1.96 | 1.86 | 1.88 |
| (mmol/L) | | | | | | | | |
| Difference | 0.12 | 0.66 | 0.57 | 0.50 | 0.23 | −0.12 | 0.00 | −0.05 |
| (mmol/L) | | | | | | | | |
| P-value | 0.378 | <0.001 | <0.001 | <0.001 | 0.061 | 0.301 | 0.984 | 0.687 |
| (pairwise) | | | | | | | | |

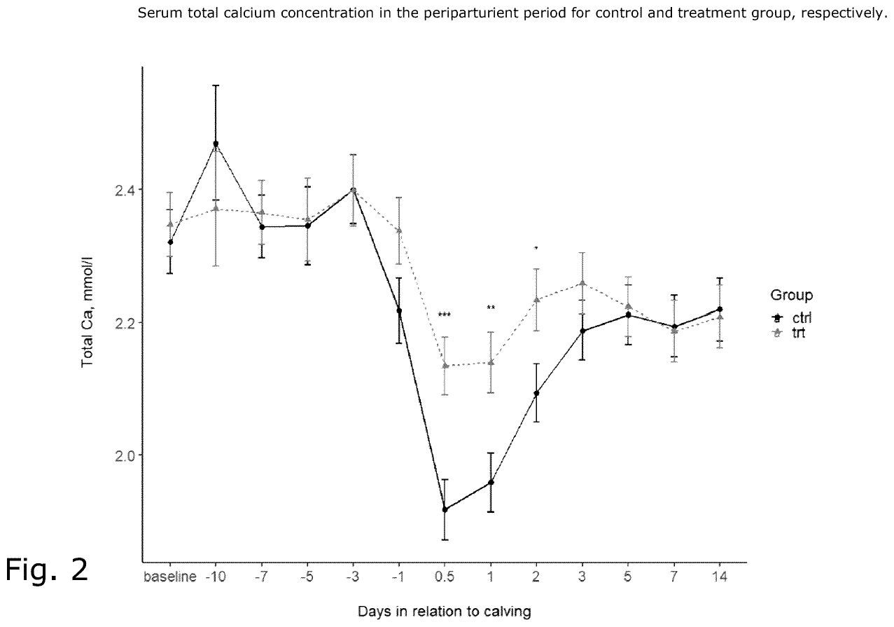

The experimental group had significantly (P<0.001) higher concentration of serum total calcium at 12 hours postpartum, and at day 1 and day 2 after calving (Table 4a & 4b and FIG. 2). The calcium concentration in the blood was on these days, on average, 0.22, 0.18 and 0.14 mmol/L, respectively, higher than that of control cows. This demonstrates that supplementing a phosphate binder in the last 21 days until calving period successfully reduced the subclinical hypocalcaemia in the periparturient cow.

Table 4a & 4b. Serum total calcium concentration (mmol/L) in the periparturient period for control and treatment group. The standard error of the mean (SEM) for the entire control group was 0.05 and for the entire treatment group was 0.05. SEM difference between the entire control group and the entire treatment group was 0.06. P-value shown for contrast test between control and treatment.

TABLE 4a

| | Day from calving | | | | | |
|---|---|---|---|---|---|---|
| | baseline | −10 | −7 | −5 | −3 | −1 |
| Control (mmol/L) | 2.32 | 2.47 | 2.34 | 2.35 | 2.40 | 2.22 |
| Treatment | 2.35 | 2.37 | 2.37 | 2.35 | 2.40 | 2.34 |
| (mmol/L) | | | | | | |
| Difference | −0.03 | 0.10 | −0.02 | −0.01 | 0.00 | −0.12 |
| (mmol/L) | | | | | | |
| P-value (pairwise) | 0.7015 | 0.415 | 0.760 | 0.913 | 0.986 | 0.085 |

TABLE 4b
(continued from table 4a).

| | Day from calving | | | | | | | |
|---|---|---|---|---|---|---|---|---|
| | baseline | 0.5 | 1 | 2 | 3 | 5 | 7 | 14 |
| Control | 2.32 | 1.92 | 1.96 | 2.09 | 2.19 | 2.21 | 2.19 | 2.22 |
| (mmol/L) | | | | | | | | |
| Treatment | 2.35 | 2.14 | 2.14 | 2.23 | 2.26 | 2.22 | 2.19 | 2.21 |
| (mmol/L) | | | | | | | | |
| Difference | −0.03 | −0.22 | −0.18 | −0.14 | -0.07 | -0.01 | 0.01 | 0.01 |
| (mmol/L) | | | | | | | | |
| P-value | 0.7015 | <0.001 | 0.005 | 0.028 | 0.268 | 0.852 | 0.903 | 0.974 |
| (pairwise) | | | | | | | | |

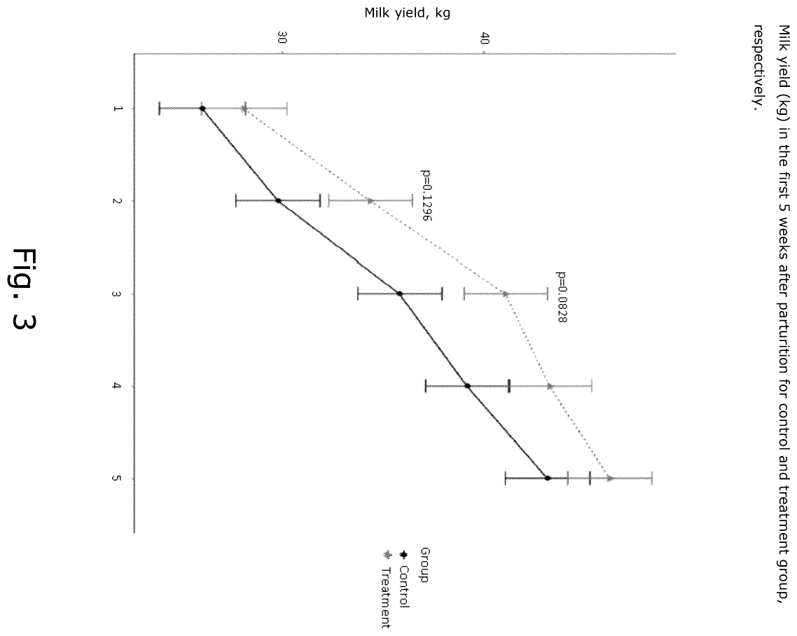

Daily milk yield tended to be higher in the treatment group (Table 5 & FIG. 3). The higher milk yield is often associated with a lower frequency of subclinical hypocalcemia.

Table 5. Milk yield (kg/day) and standard error of the mean (SEM) for the first 5 weeks after parturition for control and treatment group. P-value shown for contrast test between control and treatment.

| | Week from calving | | | | | |
|---|---|---|---|---|---|---|
| | 1 | 2 | 3 | 4 | 5 | SEM |
| Control (kg/day) | 26.0 | 29.8 | 35.8 | 39.2 | 43.2 | 2.1 |
| Treatment (kg/day) | 28.1 | 34.3 | 41.1 | 43.3 | 46.3 | 2.1 |
| Difference (kg/day) | −2.1 | −4.6 | −5.3 | −4.1 | −3.1 | |
| P-value (pairwise) | | 0.492 | 0.130 | 0.083 | 0.174 | |

The experimental group had lower frequency of subclinical hypocalcemia (Ca<2.1 mmol/L) (Table 6). In the control group 63 percent of animals were diagnosed as being subclinical hypocalcemic at 12 hours after calving, which is 3 times higher than the level in the treatment group (22%). At 24 and 48 hours after calving the control group continued to have a higher level of subclinical hypocalcemia than the treatment group.

Table 6. The percentage of subclinical hypocalcemia defined as plasma calcium concentration below 2.1 mmol/L at 12, 24 and 48 hours from calving for the control and treatment group, respectively.

| | Subclinical hypocalcemia (Ca < 2.1 mmol/L) | |
|---|---|---|
| Hours after calving | Control | Treatment |
| 12 hours after calving | 63% | 22% |
| 24 hours after calving | 41% | 19% |
| 48 hours after calving | 29% | 19% |

1.-16. (canceled)

17. A composition comprising one or more phosphate-binders selected from aluminium, iron, sevelamer, and lanthanum salts with inorganic or organic acids, a hydrate thereof or any mixture thereof for use before parturition in the prophylactic treatment of parturient hypocalcemia in a ruminant animal.

18. The composition for use according to claim 17, wherein the inorganic or organic acids are selected from the group consisting of hydrochloric acid, hydrobromic acid, hydroiodic acid, sulfuric acid, nitric acid, acetic acid, carbonic acid, and citric acid.

19. The composition for use according to claim 17, wherein the one or more phosphate-binders are selected from the group consisting of aluminium citrate, aluminium chloride, aluminium carbonate, aluminium sulphate, aluminium oxide, aluminium ammonium sulphate, aluminium sodium sulphate, aluminium potassium sulphate, aluminium calcium silicate, aluminium silicate, aluminium hydroxide, aluminium bromide, aluminium iodide, aluminium nitrate, sodium aluminium phosphate, lanthanum carbonate, lanthanum carbonate hydroxide, lanthanum oxycarbonate, lanthanum chloride, ferrous citrate, ferrous sulphate, ferrous citrate, ferrous chloride, ferrous carbonate, ferrous bicarbonate, ferric citrate, ferric sulphate, ferric chloride, ferric ammonium citrate, sucroferric oxyhydroxide, ferric trimaltol, ferric bicarbonate, ferric carbonate, sevelamer hydrochloride, sevelamer carbonate, a hydrate thereof or any mixture thereof.

20. The composition for use according to claim 17, wherein the one or more phosphate-binders are selected from the group consisting of aluminium citrate, aluminium chloride, aluminium sulphate, lanthanum chloride, lanthanum carbonate hydroxide, lanthanum oxycarbonate, ferrous citrate, ferrous chloride, ferrous carbonate, ferrous bicarbonate, ferrous sulphate, ferric citrate, ferric sulphate, ferric chloride, ferric ammonium citrate, sevelamer hydrochloride, sevelamer carbonate, or selected from the group consisting of lanthanum chloride, aluminium chloride, aluminium sulphate, ferrous sulphate, sevelamer hydrochloride, lanthanum carbonate, a hydrate thereof or a mixture thereof.

21. The composition for use according to claim 17, wherein the ruminant animal is selected from the group consisting of cows, sheep, and goats.

22. The composition for use according to claim 17 for use in the period of the last 4 weeks before parturition, such as at least in the period 1-10 days before parturition.

23. The composition for use according to claim 17, wherein the composition further comprises a zeolite.

24. The composition for use according to claim 23, wherein the weight ratio of the one or more phosphate-binders to the zeolite is in the range of 99:1 to 10:90.

25. The composition for the use according to claim 17, further one or more nutrients selected from minerals, vitamins and amino acids.

26. The composition according to claim 25, wherein the minerals, vitamins and amino acids are selected from choline, magnesium oxide, magnesium chloride, magnesium sulphate, sodium chloride, sodium bicarbonate, sodium sulphate, selenium chloride, sodium selenite, cobalt chloride, cobalt sulphate, iodine chloride, copper chloride, copper sulphate, manganese chloride, manganese sulphate, manganese oxide, zinc chloride, zinc sulphate, zinc oxide, vitamin A, vitamin D, vitamin E, vitamin B12, pantothenic acid, folic acid, thiamine, biotin, niacin, and lysine, isoleucine, threonine, lysine and rumen protected methionine, respectively.

27. The composition according to any claim 25, wherein the one or more phosphate-binders are selected from the group consisting of aluminium citrate, aluminium chloride, aluminium carbonate, aluminium sulphate, aluminium oxide, aluminium ammonium sulphate, aluminium sodium sulphate, aluminium potassium sulphate, aluminium calcium silicate, aluminium silicate, aluminium hydroxide, aluminium bromide, aluminium iodide, aluminium nitrate, sodium aluminium phosphate, lanthanum carbonate, lanthanum carbonate hydroxide, lanthanum oxycarbonate, lanthanum chloride, ferrous citrate, ferrous sulphate, ferrous citrate, ferrous chloride, ferrous carbonate, ferrous bicarbonate, ferric citrate, ferric sulphate, ferric chloride, ferric ammonium citrate, sucroferric oxyhydroxide, ferric trimaltol, ferric bicarbonate, ferric carbonate, sevelamer hydrochloride, sevelamer carbonate, a hydrate thereof or any mixture thereof, such as selected from the group consisting of aluminium citrate, aluminium chloride, aluminium sulphate, lanthanum chloride, lanthanum carbonate hydroxide, lanthanum oxycarbonate, ferrous citrate, ferrous chloride, ferrous carbonate, ferrous bicarbonate, ferrous sulphate, ferric citrate, ferric sulphate, ferric chloride, ferric ammonium citrate, sevelamer hydrochloride, sevelamer carbonate, or selected from the group consisting of lanthanum chloride, aluminium chloride, aluminium sulphate, ferrous sulphate, sevelamer hydrochloride, lanthanum carbonate, a hydrate thereof or a mixture thereof, optionally in combination with one or more zeolites, e.g. a synthetic sodium aluminosilicate zeolite A type.

28. A composition comprising one or more phosphate-binders selected from aluminium, iron, sevelamer, and lanthanum salts with inorganic or organic acids, a hydrate thereof or any mixture thereof for use before parturition in the prophylactic treatment of parturient hypocalcemia in a ruminant animal, e.g. a cow,
wherein the composition is in unit dosage form(s) each comprising 100-800 g of the phosphate-binder, such as 150-700, such as 200-600, such as 250-500, such as 300-400 g of the phosphate binder, g of the phosphate-binder(s), and
wherein one or more dosages per day of the unit dosages of the composition are manually administered to the gastrointestinal tract of the ruminant animal.

29. The composition according to claim 28, wherein the composition further comprises one or more nutrients selected from minerals, vitamins and amino acids.

30. The composition according to claim 28, wherein one or more phosphate-binders are selected from the group consisting of aluminium citrate, aluminium chloride, aluminium carbonate, aluminium sulphate, aluminium oxide, aluminium ammonium sulphate, aluminium sodium sulphate, aluminium potassium sulphate, aluminium calcium silicate, aluminium silicate, aluminium hydroxide, aluminium bromide, aluminium iodide, aluminium nitrate, sodium aluminium phosphate, lanthanum carbonate lanthanum carbonate hydroxide, lanthanum oxycarbonate, lanthanum chloride, ferrous citrate, ferrous sulphate, ferrous chloride, ferrous carbonate, ferrous bicarbonate, ferric citrate, ferric sulphate, ferric chloride, ferric ammonium citrate, sucroferric oxyhydroxide, ferric trimaltol, ferric bicarbonate, ferric carbonate, sevelamer hydrochloride, sevelamer carbonate, a hydrate thereof or any mixture thereof, such as selected from the group consisting of aluminium citrate, aluminium chloride, aluminium sulphate, lanthanum chloride, lanthanum carbonate hydroxide, lanthanum oxycarbonate, ferrous citrate, ferrous chloride, ferrous carbonate, ferrous bicarbonate, ferrous sulphate, ferric citrate, ferric sulphate, ferric chloride, ferric ammonium citrate, sevelamer hydrochloride, sevelamer carbonate, or selected from the group consisting of lanthanum chloride, aluminium chloride, aluminium sulphate, ferrous sulphate, sevelamer hydrochloride, lanthanum carbonate, a hydrate thereof or a mixture thereof, optionally in combination with one or more zeolites, e.g. a synthetic sodium aluminosilicate zeolite A type.

31. A composition comprising one or more phosphate-binders selected from aluminium, iron, sevelamer, and lanthanum salts with inorganic or organic acids, a hydrate thereof or any mixture thereof for use in the prophylactic treatment of parturient hypocalcemia in a ruminant animal, e.g. a cow,
wherein the composition is presented to the animal in wet form comprising 15-80% by weight of water.

32. The composition according to claim 31, wherein one or more phosphate-binders are selected from the group consisting of aluminium citrate, aluminium chloride, aluminium carbonate, aluminium sulphate, aluminium oxide, aluminium ammonium sulphate, aluminium sodium sulphate, aluminium potassium sulphate, aluminium calcium silicate, aluminium silicate, aluminium hydroxide, aluminium bromide, aluminium iodide, aluminium nitrate, sodium aluminium phosphate, lanthanum carbonate, lanthanum carbonate hydroxide, lanthanum oxycarbonate, lanthanum chloride, ferrous citrate, ferrous sulphate, ferrous citrate, ferrous chloride, ferrous carbonate, ferrous bicarbonate, ferric citrate, ferric sulphate, ferric chloride, ferric ammonium citrate, sucroferric oxyhydroxide, ferric trimaltol, ferric bicarbonate, ferric carbonate, sevelamer hydrochloride, sevelamer carbonate, a hydrate thereof or any mixture thereof, such as selected from the group consisting of aluminium citrate, aluminium chloride, aluminium sulphate, lanthanum chloride, lanthanum carbonate hydroxide, lanthanum oxycarbonate, ferrous citrate, ferrous chloride, ferrous carbonate, ferrous bicarbonate, ferrous sulphate, ferric citrate, ferric sulphate, ferric chloride, ferric ammonium citrate, sevelamer hydrochloride, sevelamer carbonate, or selected from the group consisting of lanthanum chloride, aluminium chloride, aluminium sulphate, ferrous sulphate, sevelamer hydrochloride, lanthanum carbonate, a hydrate thereof or a mixture thereof, optionally in combination with one or more zeolites, e.g. a synthetic sodium aluminosilicate zeolite A type.
